(19) United States
(12) Patent Application Publication
(10) Pub. No.: US 2026/0083545 A1
(43) Pub. Date: Mar. 26, 2026

(54) APPARATUS FOR TREATMENT OF AORTA DISEASE WITH A BALLOON EXPANDABLE SECTION

(71) Applicant: Braile Biomédica Indústria Comércio e Representações Ltda., São José do Rio Preto (BR)

(72) Inventors: Rafael Braile Cunha, São José do Rio Preto (BR); Vinicius Ladeia Semenzim, São José do Rio Preto (BR); Marco Orlandi, Sorisole (IT); Antonio Rizza, Pisa (IT); Lucas Roberto Dos Santos, São José do Rio Preto (BR)

(73) Assignee: Braile Biomédica Indústria Comércio e Representações Ltda., São José do Rio Preto, SP (BR)

(21) Appl. No.: 19/304,192

(22) Filed: Aug. 19, 2025

Related U.S. Application Data
(60) Provisional application No. 63/687,885, filed on Aug. 28, 2024.

(51) Int. Cl. A61F 2/07

(52) U.S. Cl. CPC A61F 2/07; A61F 2210/0004; A61F 2210/0014; A61F 2220/0008; A61F 2220/0075; A61F 2230/0006; A61F 2250/0098

(57) ABSTRACT

A stent graft apparatus for endovascular treatment of aortic diseases includes a balloon-expandable section and a self-expanding section connected by radial sutures to form a unitary device. The balloon-expandable section comprises a metallic framework configured for deployment via balloon inflation and includes a plurality of radially equidistant radiopaque markers for fluoroscopic visualization. The self-expanding section comprises a shape-memory material configured to exert continuous radial force upon release. The connected sections are sequentially deployed through a catheter-based delivery system, allowing the balloon-expandable section to anchor the graft precisely at the treatment site before the self-expanding section is released to conform to the vessel anatomy. This configuration facilitates both accurate positioning and anatomical adaptability, reducing the risk of migration and improving fixation in complex aortic regions. The apparatus may be used to treat aneurysms, dissections, or injuries in various segments of the aorta.

### CROSS-REFERENCE TO RELATED APPLICATIONS

The present application claims priority to U.S. Provisional Application No. 63/687,885 filed Aug. 28, 2024, titled “APPARATUS FOR TREATMENT OF AORTA DISEASE WITH A BALLOON-EXPANDABLE SECTION,” which is hereby incorporated by reference in its entirety.

### TECHNICAL FIELD

The embodiments generally relate to the technical field of endovascular stent grafts for treating aortic diseases.

### BACKGROUND

Conventional endovascular stent graft systems are commonly used to treat aortic diseases such as aneurysms, dissections, and traumatic injuries. These systems typically include self-expanding or balloon-expandable stent grafts that are delivered to the target site via a catheter-based delivery system. Self-expanding stent grafts, often made from shape-memory alloys like nitinol, automatically expand to engage the vessel wall upon release from a delivery sheath. Balloon-expandable stent grafts, on the other hand, rely on an inflatable balloon to mechanically expand the graft at the desired location, providing controlled placement.

Although these systems have been widely adopted in clinical practice, they may present certain challenges in specific anatomical regions. In tortuous or highly curved segments of the aorta, such as the aortic arch, it can be difficult to achieve both accurate deployment and conformability to the vessel wall. Balloon-expandable systems provide precise placement but may offer limited adaptability to varying vessel geometries. Conversely, self-expanding systems conform well to anatomy but may experience positional shifts during deployment. Delivery systems must also accommodate a range of vessel sizes and contours while minimizing the risk of endoleaks, graft migration, or injury to the vessel wall.

### SUMMARY

This summary is provided to introduce a variety of concepts in a simplified form that is further disclosed in the detailed description of the embodiments. This summary is not intended to identify key or essential inventive concepts of the claimed subject matter, nor is it intended to determine the scope of the claimed subject matter.

The present disclosure provides a stent graft apparatus for the endovascular treatment of aortic diseases, incorporating a hybrid structure that combines a balloon-expandable section with a self-expandable section. The balloon-expandable section includes a metallic structure configured to expand in response to balloon inflation, enabling precise positioning and anchoring of the stent graft within the target aortic region. A plurality of fixed radiopaque markers is distributed radially on the balloon-expandable section to facilitate accurate visualization and deployment under fluoroscopic guidance.

Connected to the balloon-expandable section via radial or longitudinal sutures is a self-expandable section formed from a material that exerts continuous radial force against the vessel wall. This configuration enhances fixation and conformance to curved or tortuous anatomies, such as the aortic arch, where conventional single-mode grafts may struggle to maintain optimal position or vessel wall apposition. The radial or longitudinal sutures join the two sections and ensure structural integrity and coordinated deployment between the two sections, allowing the apparatus to be delivered as a unit through a catheter-based system.

By enabling sequential deployment, first expanding the balloon-expandable segment for precise placement, followed by automatic expansion of the self-expandable segment, the apparatus addresses the challenge of both positioning accuracy and anatomical adaptability in complex aortic regions. This hybrid configuration reduces the likelihood of graft migration and improves overall fixation performance during and after deployment.

Other illustrative variations within the scope of the invention will become apparent from the detailed description provided hereinafter. The detailed description and enumerated variations, while disclosing optional variations, are intended for purposes of illustration only and are not intended to limit the scope of the invention.

### BRIEF DESCRIPTION OF THE DRAWINGS

A more complete understanding of the embodiments, and the attendant advantages and features thereof, will be more readily understood by references to the following detailed description when considered in conjunction with the accompanying drawings wherein:

FIG. 1 illustrates a side view of the hybrid stent graft (or endoprosthesis) for aorta disease treatment, according to the disclosed embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing exemplary embodiments in detail, it is noted that the embodiments reside primarily in combinations of components related to devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

A stent graft apparatus may be configured for endovascular treatment of aortic diseases, including aneurysms, dissections, and traumatic vascular injuries. The apparatus may include a hybrid expandable structure comprising a balloon-expandable section and a self-expandable section joined by, for example, radial or longitudinal suturing. The hybrid configuration may allow for both precise placement and conformable anchoring within complex aortic anatomies.

The balloon-expandable section may include a metallic framework, such as a stent-like cylindrical structure, that is plastically deformable upon inflation of an angioplasty balloon. This section may be fabricated from a metal such as stainless steel, cobalt-chromium alloy, or other materials that retain a deployed configuration after balloon expansion. The balloon-expandable section may serve as the proximal portion of the stent graft and may be adapted to anchor the device at the target treatment site. A plurality of radiopaque markers may be affixed to the balloon-expandable section. These markers may be constructed from materials such as gold, platinum, or tantalum and may be arranged radially equidistant to allow visualization of the device under fluoroscopy. In some embodiments, fixation posts may also be integrated into the balloon-expandable section to enhance initial mechanical anchoring upon expansion.

The self-expandable section may comprise a tubular framework of shape-memory material, such as nitinol, capable of undergoing radial expansion when released from a constraining sheath. This section may form the distal portion of the stent graft and may be configured to apply a continuous outward radial force against the aortic wall after deployment. The self-expanding section may be constructed from interconnected stent rings or lattice structures designed to flexibly conform to tortuous or irregular vessel geometries.

The balloon-expandable and self-expandable sections may be connected through a set of joining sutures, such as radial or longitudinal sutures. The sutures may be formed from biocompatible thread or filament, such as polyester or PTFE, and may be distributed circumferentially to evenly distribute mechanical forces during deployment. The sutured junction may ensure mechanical continuity between the two expandable zones while maintaining flexibility during navigation and placement.

The stent graft may be configured for delivery using a catheter-based system. The system may include an outer sheath and an inner guide catheter, with the stent graft loaded in a compressed state. A deployment sequence may involve positioning the catheter system within the aorta using standard endovascular access techniques. Once in position, the balloon-expandable section may be deployed first by retracting the sheath and inflating a balloon situated coaxially within the metallic structure. The balloon inflation may plastically expand the balloon-expandable section and secure it at the target site. Following balloon deflation and withdrawal, the sheath may be further retracted to release the self-expandable section, which then automatically expands to engage the aortic wall.

The delivery system may include a control handle to allow for sequential or staged release of the two sections. The hybrid deployment approach may offer improved precision by anchoring the balloon-expandable section first, thereby reducing migration or malposition of the self-expanding segment during final release.

In some configurations, the stent graft may include features such as heparin coatings to reduce thrombogenicity, or bioresorbable sutures that degrade after sufficient endothelialization. The device may be manufactured in various lengths and diameters to accommodate different segments of the aorta, including the aortic arch, descending thoracic aorta, and abdominal aorta. The hybrid stent graft may be preloaded in a sterile delivery system and supplied in a compressed, ready-to-deploy configuration.

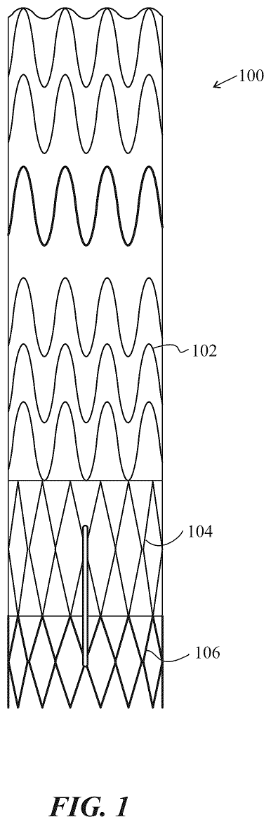

FIG. 1 illustrates a side view of the hybrid stent graft 100 (or endoprosthesis) for aorta disease treatment, according to the disclosed embodiments. The endoprosthesis contains two expansion systems. The proximal region includes a balloon-expandable stent section 104 connected to a self-expanding section 102. In this way, the balloon-expandable section 104 enables controlled and precise placement by expanding in response to balloon inflation, which is particularly useful in anatomically sensitive or high-flow regions such as the aortic arch. Once anchored, the self-expanding section 102, which is attached via joining sutures, such as radial or longitudinal sutures, may be deployed to conform to the vessel wall and maintain continuous radial force, promoting stable fixation and improved sealing across tortuous or curved anatomies. This sequential deployment approach reduces the risk of device migration during the procedure and enhances positional accuracy. Integrating both expansion mechanisms into one unified structure also allows the graft to be delivered and deployed through a single catheter system, eliminating the need for overlapping multiple devices and reducing procedural complexity. Additionally, the combination of immediate mechanical fixation from the balloon-expandable portion and long-term conformability from the self-expanding portion supports stable anchoring in dynamic aortic environments, helping to minimize complications such as endoleaks or incomplete wall apposition.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

It will be appreciated by persons skilled in the art that the present embodiment is not limited to what has been particularly shown and described hereinabove. A variety of modifications and variations are possible considering the above teachings without departing from the following claims.

I/We claim:

1. An apparatus for endovascular treatment of aortic disease, comprising:
a stent graft including a proximal balloon-expandable section and a distal self-expandable section,
wherein the balloon-expandable section comprises a metallic structure configured to expand in response to an inflatable balloon;
wherein the self-expandable section comprises a self-expanding framework configured to exert a radial outward force upon deployment;
wherein the balloon-expandable section and the self-expandable section are integrally connected via radial or longitudinal sutures to form a continuous unitary device; and
wherein the apparatus comprises a plurality of radiopaque markers fixedly attached to the balloon-expandable section and distributed radially equidistantly.

2. The apparatus of claim 1, wherein the metallic structure of the balloon-expandable section comprises stainless steel or cobalt-chromium alloy.

3. The apparatus of claim 1, wherein the self-expanding framework comprises a nitinol structure.

4. The apparatus of claim 1, wherein the radiopaque markers are composed of at least one of gold, platinum, or tantalum.

5. The apparatus of claim 1, wherein the sutures are arranged in a circumferential pattern to evenly distribute mechanical stress during deployment.

6. The apparatus of claim 1, wherein the balloon-expandable section is adapted to remain fixed in position during deployment of the self-expandable section.

7. An endoprosthesis apparatus for treating aortic artery disease, comprising:
a delivery catheter;
a stent graft navigable through the catheter, the stent graft including:
a proximal balloon-expandable metallic segment configured for precise positioning via balloon inflation;
a distal self-expandable segment configured to automatically expand upon deployment;
a set of radial or longitudinal sutures joining the balloon-expandable segment to the self-expandable segment; and
a plurality of radiopaque markers affixed to the balloon-expandable segment and visible under fluoroscopy to facilitate precise placement, wherein the balloon-expandable segment is deployed prior to the self-expandable segment.

8. The apparatus of claim 7, wherein the catheter comprises a sheath configured to retract sequentially to release the balloon-expandable segment, followed by the self-expandable segment.

9. The apparatus of claim 7, wherein the stent graft is pre-loaded in a compressed state within the catheter prior to navigation through the vasculature.

10. The apparatus of claim 7, wherein the balloon is positioned coaxially within the balloon-expandable segment and is adapted for selective inflation.

11. The apparatus of claim 7, wherein the radiopaque markers are placed at the ends and midpoint of the balloon-expandable segment for enhanced visualization.

12. The apparatus of claim 7, wherein the delivery catheter comprises a handle mechanism operable to control sequential deployment of the two sections.

13. A hybrid stent graft apparatus for endovascular treatment of aneurysms, dissections, or injuries in the aorta, comprising:
a proximal balloon-expandable section configured to be positioned at a target region and anchored through balloon expansion;
a distal self-expandable section configured to conform to the patient's anatomy and maintain fixation through radial force;
a plurality of radially distributed and equidistant radiopaque markers affixed to the balloon-expandable section; and
a radial or longitudinal suturing interface joining the balloon-expandable and self-expandable sections enabling structural continuity and synchronized deployment, wherein the apparatus is adapted for sequential deployment such that the balloon-expandable section is deployed and anchored before release of the self-expandable section.

14. The apparatus of claim 13, wherein the self-expandable section is configured to expand to a preset diameter upon release from the catheter.

15. The apparatus of claim 13, wherein the balloon-expandable section is configured to withstand high-pressure balloon inflation without permanent deformation.

16. The apparatus of claim 13, wherein the apparatus is sized to conform to a curvature radius characteristic of the aortic arch.

17. The apparatus of claim 13, wherein the balloon-expandable section comprises at least three fixation posts to prevent migration.

18. The apparatus of claim 13, wherein the self-expandable section comprises multiple interconnected stent rings for enhanced flexibility.

19. The apparatus of claim 13, wherein the radial or longitudinal sutures are bioresorbable.

20. The apparatus of claim 13, wherein the apparatus further comprises a heparin coating to reduce thrombogenicity.
